(19) United States
(12) Patent Application Publication
(10) Pub. No.: US 2009/0187148 A1
(43) Pub. Date: Jul. 23, 2009

(54) Closed cover for intravenous therapy equipment ports

(76) Inventor: Keisha Gay Knight, Hackettstown, NJ (US)

Correspondence Address: Keisha G. Knight, 4 Winding Hill Drive, Hackettstown, NJ, 07840, US

(21) Appl. No.: 12/319,459

(22) Filed: Jan. 7, 2009

Related U.S. Application Data
(63) Continuation of application No. 61/010,430, filed on Jan. 8, 2008.

(51) Int. Cl. A61B 19/02

(52) U.S. Cl. 604/164.08

(57) ABSTRACT

A closed cover for intravenous therapy equipment ports', used for the prevention of cross contamination, made by the collection of airborne particles that collect on the surface openings of rubber ports. Ports are direct openings on Intravenous therapy equipments that supply a direct access to patients' circulatory system. Keeping intravenous therapy equipments' rubber ports surface covered when not in use, reduces the risk of cross contamination by foreign particles which carries microorganisms that cause disease.

This application claims priority to provisional application No. 61/010,430

A closed cover for intravenous therapy equipment ports', used for the prevention of cross contamination, made by the collection of airborne foreign particles that collect on the surface opening of the rubber ports. Ports are direct openings on Intravenous therapy equipment that supply a direct access to patients' circulatory system.

Closed cover may be composed of any suitable materials, such as plastic, synthetic resin, metal, rubber etc., which will be impervious to moisture, air and air particles. The cover having a flat closed circular head (top), connected to an hollow extended circular neck area (middle), and the base (bottom)-hollow stretchable circular area with inner flanges which allows cover to fit over the body of the port and latching to under ports' body to keep it in place and snapping to base ring for further closure. Cover is connected to intravenous tubing by way of a connector (plastic etc.), which holds cover in place at one end and is then connected directly to intravenous equipment (tubing) or connected to a circular base ring, placed around the tubing during manufacture of equipment prior to rubber port being applied; thus keeping the base ring in place. Base ring is made to allow cover to snap over its' mouth area to close, and a simple push-up to release cover for use of port.

### BACKGROUND OF THE INVENTION

The invention relates to covers for intravenous therapy equipments and more particularly to a closed cover which shields the rubber ports surface from cross contamination, made by the collection of foreign air particles which may carry harmful pathogens that collect on the surface of ports. Therefore, decreasing the risk of infection by cross contamination to patient.

The foreign matters include those which are retained on the rubber ports' surface even after washing. When the surface of a washed rubber port is observed by a scanning electron microscope at magnification of about 10.sup 0.2, -10.sup 0.3 times, the presence of indented locations or attachments of solid foreign matters can be observed there on. It seems that they are connected to an increase in the number of foreign matters with time. Prolong use of injecting hypodermic needles into equipments rubber surface, will result in surface rubber plug deterioration, allowing for contamination.

Microorganisms that cause disease are transmitted by a number of routes, and the same microorganisms can be transmitted by more than one route. Airborne, contact, food transmission etc., are the most common vehicles that carry microorganisms that can lead to cross contamination to intravenous therapy equipments. Nosocomial and exogenous infections, are caused by organisms that enter the patient from the environment, other patients, staff members, inanimate objects, equipment used in intravenous therapy and other fixtures.

### SUMMARY OF THE INVENTION

The present invention teaches a closed cover which fits over intravenous therapy equipments' rubber port and snaps down and latches to under ports' head and to base ring to close. The closed cover keeps a constant shield over intravenous therapy equipments' rubber port surface, when port is not in use, used for administering intravenous therapy directly to patients' circulatory system, preventing any cross contamination by microorganisms to equipment. Such cover is made of material which is impervious to moisture, air and air particles and is easily removed by a “push-up” with finger for removal of cover. A flat closed circular top, connected to hollow extended neck area, connected to the circular hollow stretchable base area with inner flanges at the bottom, which allows for cover to fit and latch on to under the rubber port's body and snapping to base ring to close.

Keeping intravenous therapy equipments' rubber port surface covered when not in use, reduces the risk of cross contamination by foreign particles which can carry microorganism that can cause disease and infect patient.

### REFERENCES CITED

U.S. Patent Documents

| | | |
|---|---|---|
| 4,671,331 | Jun. 9, 1987 | Pruden |
| 2,364,126 | December 1944 | Cantor et al. |
| 1,189,465 | July 1916 | Mayo |
| 2,524,365 | October 1950 | Smith |
| 2,186,888 | January 1940 | Tullar et al. |

### Books:

Microbiology—Principles & Applications—Joan G. Creager, Jacqueline G. Black, Vee E. Davison 1990
Clinical Nursing Skills—Sandra f. Smith, RN, MS, Donna J. Duell, RN, MS Third Edition
Introduction to Nursing—Barbara Kozier, Glenora Erb, Patricia Mckay Bufalino 1989

### BRIEF DESCRIPTION OF THE DRAWINGS

The foregoing and other objects, features and advantages of the invention will become more apparent by reference to the accompanying drawings and the following detailed description taken in conjunction with accompanying drawings in which:

FIG. 1 is a perspective view of a cover with a connector and base ring.

FIG. 2 illustrates a cover with connector and base ring around intravenous therapy equipment.

FIG. 3 is a perspective view of a cover, covering ports' body, inner base flanges holding ports' body in place and snapped on to circular base ring. A connector connects cover to circular base ring.

### DETAILED DESCRIPTION

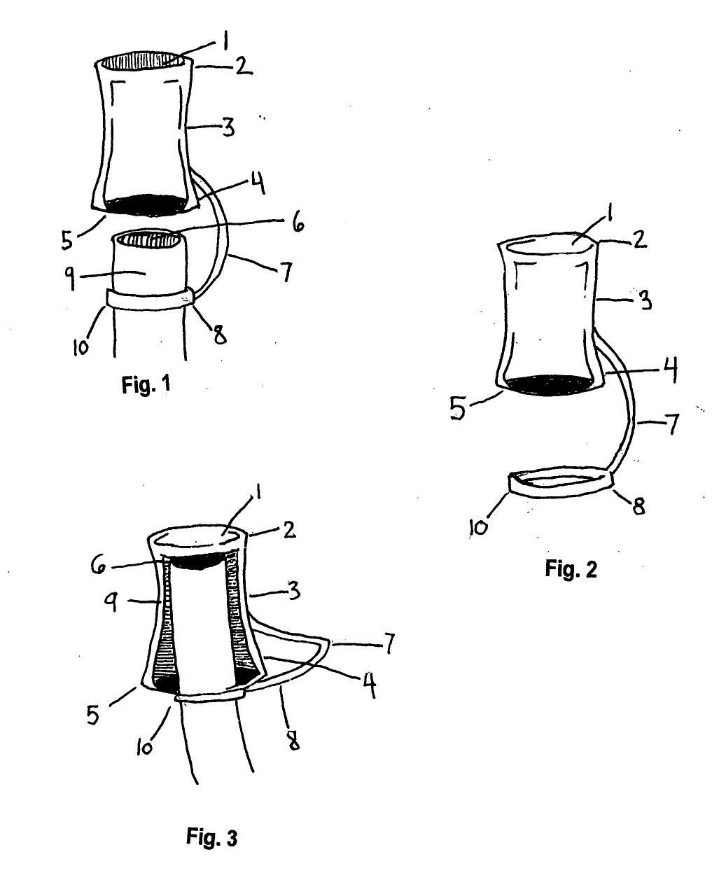

Reference is first made to FIG. 1, which illustrates an intravenous therapy equipment port cover (2), with a connector (7), connecting the cover (2), to the circular ring base (8). Cover (2), is made of a flat closed top (1), which connects to a hollow extended neck area (3) and a stretchable base area (4) with inner flanges (5) at the bottom. Circular base ring (8), has a outward mouth piece area (10), which allows cover (2) to snap over mouth piece (10) to close.

FIG. 2. Cover (2) is viewed above intravenous therapy rubber ports' body (9), with connector (7), connecting cover (2) to circular base ring (8), which is placed around Intravenous therapy equipment (13), during manufacture.

FIG. 3 is a perspective view of a closed cover (2), covering ports' body (9), with inner flanges (5), latching on to under ports' body (9), holding port (9) in place and snapping to over circular base rings' mouth piece (10) to close; thus shielding the surface opening of port (6), from contamination by the collection of airborne foreign particles.

While the principals of the invention have now been made clear in an illustrative form, there will become obvious to those skilled in the art many modifications in structure, arrangements, portions and materials used in the practice of the invention and otherwise which are particularly adapted for specific operating requirements without departing from those principles. The appended claims are therefore intended to cover and embrace any such modifications within the limits only of the true spirit and scope of the invention.

I claim:

1. A cover for use on intravenous therapy equipment port's. Ports are direct openings on Intravenous therapy equipments that supply a direct access to patients' circulatory system.
a closed non-pierce able circular top impervious to air and air particles adapted for easy grasping and proper handling of said equipment.
a hollow circular base adapted to fit over said port, with inner flanges which are designed to latch to under said port's head, to keep it in place.
a hollow narrow circular neck area which connects said closed top with said base.

2. A cover as set forth in claim 1 wherein said cover is made of materials that aids in preventing and stopping cross contamination of foreign microorganisms to surface of said ports.

3. A cover as set forth in claim 1 wherein said cover is held up by way of a connector, which holds cover close to port's opening.

4. A cover as set forth in claim 3 wherein said connecter is placed around and under said intravenous therapy equipment port's, during manufacture of equipment.

5. A cover as set forth in claim 1 wherein said cover is made disposable. After use of the port, a new disposable cover is placed each time.

6. A cover as set forth in claim 1 wherein said cover is made and packaged sterile.
